(19) United States
(12) Patent Application Publication
(10) Pub. No.: US 2020/0376273 A1
(43) Pub. Date: Dec. 3, 2020

(54) ELECTROGRAPHIC OBSESSION IN HUMAN NUCLEUS ACCUMBENS

(71) Applicant: The Board of Trustees of the Leland Stanford Junior University, Stanford, CA (US)

(72) Inventors: Casey H. Halpern, Menlo Park, CA (US); Kai J. Miller, Palo Alto, CA (US); Nolan R. Williams, Palo Alto, CA (US)

(21) Appl. No.: 16/888,347

(22) Filed: May 29, 2020

Related U.S. Application Data
(60) Provisional application No. 62/854,892, filed on May 30, 2019.

(51) Int. Cl. A61N 1/36; A61N 1/372; A61N 1/05

(52) U.S. Cl. CPC A61N 1/36082; A61N 1/37205; A61N 1/36196; A61N 1/36171; A61N 1/0534

(57) ABSTRACT

Provided herein are, inter alia, methods for detecting the anatomic structure of a nucleus accumbens in the brain of a subject. The method includes inserting an electrode into the ventral striatum of a subject; and detecting an oscillatory frequency of 30-40 Hz, thereby identifying the anatomic structure of a nucleus accumbens in the subject. The methods provided herein may include a step of delivering an electrical stimulation to ameliorate or prevent an OCD symptom from occurring.

### CROSS-REFERENCES TO RELATED APPLICATIONS

This application claims the benefit of U.S. Provisional Application No. 62/854,892, filed May 30, 2019, which is incorporated herein by reference in its entirety and for all purposes.

### BRIEF DESCRIPTION OF THE DRAWINGS

FIG. 1A-1D. A robust map of brain oscillations in the ventral capsule/ventral striatal region of the human brain. (FIG. 1A) Oscillations of different frequency are plainly visible in the raw voltage trace at three exemplar sites (+1, +8, +18 mm above target). (FIG. 1B) Power spectral densities (PSDs) on log-log axes for the three sites in (A) show clear peaks in the theta (7 Hz), alpha (9 Hz), and gamma (35 Hz) frequencies above a 1/f background shape(11). (FIG. 1C) PSDs were measured at 1 mm intervals from the cannula to target location. Colored background lines show significant oscillations (green—7 Hz/θ; blue—9 Hz/α; orange—25 Hz/b; red—35 Hz/γ). (FIG. 1D) Anatomical plotting of oscillations revealed a plain topological correspondence between each oscillation and the underlying brain anatomy. Abbreviations: sep—septal nuclei/fornix; LV—lateral ventricle; Cd—caudate; BN—bed nucleus of the stria terminalis; AC—anterior commissure; NAc—nucleus accumbens; DB—diagonal band of Broca; HTH—hypothalamus; ALIC—anterior limb of internal capsule; GP—globus pallidus; Put—putamen.

FIG. 2A-2E: Physiological changes during provocation of an obsessive fear that drives compulsive cleaning behavior. (FIG. 2A) Provocation was performed at two NAc sites (yellow/purple dots 4/6 mm ventral to the dorsal NAc border). (FIG. 2B) After resting baseline, the patient was handed a toothbrush to bring to his face and was told “imagine brushing your teeth with this dirty toothbrush”, followed by bringing his hand to his face without a toothbrush (see Supplemental video). (FIG. 2C) Action potential rate selectively increased during toothbrush-provocation at the yellow site. (‡p=1×10−6/t=5.4; †p=0.05/t=−2.0, by unpaired t-test. Error bars show S.E.M.). (FIG. 2D) PSDs reveal progressive power increase across all frequencies during the task at the yellow site. Inset axes show isolated alpha-range (8-10Hz, ‡p=2×10−6/t=5.2) and gamma-range (31-39 Hz, ‡p=7×10−7/t=5.5) amplitudes. (FIG. 2E) Conversely, a neuron captured 2 mm ventral (purple site) exhibited a significant decrease in spike rate (p=†8×10−3/t=−2.7) and gamma-range amplitude (†p=0.04/t=−2.1; ‡p=4×10−6/t=5.0) with provocation.

### DETAILED DESCRIPTION

### Definitions

Unless specifically stated or obvious from context, as used herein, the term “or” is understood to be inclusive.

Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

Ranges provided herein are understood to be shorthand for all of the values within the range.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The terms “disorder” or “disease” as provided herein are used interchangeably and refer to any deviation from the normal health of a mammal and include a state when disease/disorder symptoms are present, as well as conditions in which a deviation (e.g., chemical imbalance, infection, gene mutation, genetic defect, etc.) has occurred, but symptoms are not yet manifested or are not yet fully manifested. According to the present invention, the methods disclosed herein are suitable for use in a patient that is a member of the Vertebrate class, Mammalia, including, without limitation, primates, livestock and domestic pets (e.g., a companion animal). Typically, a patient will be a human patient.

Obsessive-compulsive disorder (OCD) as referred to herein is an anxiety disorder in which subjects have recurring, unwanted thoughts, ideas or sensations (obsessions) that cause them to do something repetitively (compulsions). The repetitive behaviors, such as for example hand washing, checking on things or cleaning, can significantly interfere with a subject's daily activities and social interactions. In subjects with OCD, thoughts are persistent and unwanted routines and behaviors are rigid and not doing them causes great distress. Subjects with OCD know or suspect their obsessions are not true; others may think they could be true (known as poor insight). Even if they know their obsessions are not true, people with OCD have a hard time keeping their focus off the obsessions or stopping the compulsive actions.

The term “obsession” or “obsessions” as referred to herein is/are recurrent and persistent thoughts, impulses, or images that cause distressing emotions such as anxiety or disgust. Many subjects with OCD recognize that the thoughts, impulses, or images are a product of their mind and are excessive or unreasonable. Yet these intrusive thoughts cannot be settled by logic or reasoning. OCD subjects may try to ignore or suppress such obsessions or offset them with some other thought or action. Exemplary obsessions include excessive concerns about contamination or harm, the need for symmetry or exactness, or forbidden sexual or religious thoughts.

The term “compulsion” or “compulsions” as provided herein refer to repetitive behaviors or mental acts that a subject feels driven to perform in response to an obsession. The behaviors are aimed at preventing or reducing distress or a feared situation. In the most severe cases, a constant repetition of rituals may fill the day, making a normal routine impossible. Compounding the anguish these rituals cause is the knowledge that the compulsions are irrational. Although the compulsion may bring some relief to the worry, the obsession returns and the cycle repeats over and over.

Conditions included in the definition of OCD provided herewith include, for example, body dysmorphic disorder (preoccupation with imagined ugliness), hypochondriasis (preoccupation with physical illness), trichotillomania (hair pulling), some eating disorders such as binge eating disorder, and neurologically based disorders such as Tourette's syndrome.

Compulsive behaviors (e.g., obsessive compulsive disorder (OCD)) as contemplated herein may include, but are not limited to, gambling characterized by an uncontrollable urge to continue gambling despite negative consequences; eating disorders, such as binge eating which is characterized by recurrent episodes of eating large quantities of food quickly and to the point of discomfort, which may be followed by feelings of depression, disgust, or guilt; night eating which is characterized by a delayed circadian pattern of food intake often accompanied by a sense of shame and/or inability to control one's eating pattern; loss of control eating which is characterized by a sense of loss of control over eating similar to that experienced in binge eating, but not necessarily accompanied by consumption of a large quantity of food; emotional or stress eating which is eating in an effort to alleviate negative emotions; compulsive eating which refers to a compulsion to overeat resulting in consumption of abnormally large quantities of food while simultaneously feeling unable to stop consumption; purge behaviors, for example self-induced vomiting, misuse of laxatives, excessive exercise; suicidal thoughts, also known as suicidal ideation, wherein an individual may consider or formulate plans to kill oneself; and suicidal attempts wherein an individual will engage in a non-fatal, self-directed injurious behavior with the intent of killing oneself

The term “nucleus accumbens” as used herein refers to a brain region in the basal forebrain located rostral to the preoptic area of the hypothalamus. The nucleus accumbens is known to play a role in brain reward circuitry.

The term “brain region(s)” is used according to its plain and ordinary meaning and refers to a brain anatomical region following standard neuroanatomy hierarchies (e.g. a functional, connective or developmental region). Exemplary brain regions include, but are not limited to, brainstem, Medulla oblongata, Medullary pyramids, Olivary body, Inferior olivary nucleus, Rostral ventrolateral medulla, Respiratory center, Dorsal respiratory group, Ventral respiratory group, Pre-Bötzinger complex, Botzinger complex, Paramedian reticular nucleus, Cuneate nucleus, Gracile nucleus, Intercalated nucleus, Area postrema, Medullary cranial nerve nuclei, Inferior salivatory nucleus, Nucleus ambiguus, Dorsal nucleus of vagus nerve, Hypoglossal nucleus, Solitary nucleus, Pons, Pontine nuclei, Pontine cranial nerve nuclei, chief or pontine nucleus of the trigeminal nerve sensory nucleus (V), Motor nucleus for the trigeminal nerve (V), Abducens nucleus (VI), Facial nerve nucleus (VII), vestibulocochlear nuclei (vestibular nuclei and cochlear nuclei) (VIII), Superior salivatory nucleus, Pontine tegmentum, Respiratory centers, Pneumotaxic center, Apneustic center, Pontine micturition center (Barrington's nucleus), Locus coeruleus, Pedunculopontine nucleus, Laterodorsal tegmental nucleus, Tegmental pontine reticular nucleus, Superior olivary complex, Paramedian pontine reticular formation, Cerebellar peduncles, Superior cerebellar peduncle, Middle cerebellar peduncle, Inferior cerebellar peduncle, Cerebellum, Cerebellar vermis, Cerebellar hemispheres, Anterior lobe, Posterior lobe, Flocculonodular lobe, Cerebellar nuclei, Fastigial nucleus, Interposed nucleus, Globose nucleus, Emboliform nucleus, Dentate nucleus, Tectum, Corpora quadrigemina, inferior colliculi, superior colliculi, Pretectum, Tegmentum, Periaqueductal gray, Parabrachial area, Medial parabrachial nucleus, Lateral parabrachial nucleus, Subparabrachial nucleus (Kölliker-Fuse nucleus), Rostral interstitial nucleus of medial longitudinal fasciculus, Midbrain reticular formation, Dorsal raphe nucleus, Red nucleus, Ventral tegmental area, Substantia nigra, Pars compacta, Pars reticulata, Interpeduncular nucleus, Cerebral peduncle, Crus cerebri, Mesencephalic cranial nerve nuclei, Oculomotor nucleus (III), Trochlear nucleus (IV), Mesencephalic duct (cerebral aqueduct, aqueduct of Sylvius), Pineal body, Habenular nucleim Stria medullares, Taenia thalami, Subcommissural organ, Thalamus, Anterior nuclear group, Anteroventral nucleus (aka ventral anterior nucleus), Anterodorsal nucleus, Anteromedial nucleus, Medial nuclear group, Medial dorsal nucleus, Midline nuclear group, Paratenial nucleus, Reuniens nucleus, Rhomboidal nucleus, Intralaminar nuclear group, Centromedial nucleus, Parafascicular nucleus, Paracentral nucleus, Central lateral nucleus, Central medial nucleus, Lateral nuclear group, Lateral dorsal nucleus, Lateral posterior nucleus, Pulvinar, Ventral nuclear group, Ventral anterior nucleus, Ventral lateral nucleus, Ventral posterior nucleus, Ventral posterior lateral nucleus, Ventral posterior medial nucleus, Metathalamus, Medial geniculate body, Lateral geniculate body, Thalamic reticular nucleus, Hypothalamus, limbic system, HPA axis, preoptic area, Medial preoptic nucleus, Suprachiasmatic nucleus, Paraventricular nucleus, Supraoptic nucleusm Anterior hypothalamic nucleus, Lateral preoptic nucleus, median preoptic nucleus, periventricular preoptic nucleus, Tuberal, Dorsomedial hypothalamic nucleus, Ventromedial nucleus, Arcuate nucleus, Lateral area, Tuberal part of Lateral nucleus, Lateral tuberal nuclei, Mammillary nuclei, Posterior nucleus, Lateral area, Optic chiasm, Subfornical organ, Periventricular nucleus, Pituitary stalk, Tuber cinereum, Tuberal nucleus, Tuberomammillary nucleus, Tuberal region, Mammillary bodies, Mammillary nucleus, Subthalamus, Subthalamic nucleus, Zona incerta, Pituitary gland, neurohypophysis, Pars intermedia, adenohypophysis, cerebral hemispheres, Corona radiata, Internal capsule, External capsule, Extreme capsule, Arcuate fasciculus, Uncinate fasciculus, Perforant Path, Hippocampus, Dentate gyrus, Cornu ammonis, Cornu ammonis area 1, Cornu ammonis area 2, Cornu ammonis area 3, Cornu ammonis area 4, Amygdala, Central nucleus, Medial nucleus (accessory olfactory system), Cortical and basomedial nuclei, Lateral and basolateral nuclei, extended amygdala, Stria terminalis, Bed nucleus of the stria terminalis, Claustrum, Basal ganglia, Striatum, Dorsal striatum (aka neostriatum), Putamen, Caudate nucleus, Ventral striatum, Striatum, Nucleus accumbens, Olfactory tubercle, Globus pallidus, Subthalamic nucleus, Basal forebrain, Anterior perforated substance, Substantia innominata, Nucleus basalis, Diagonal band of Broca, Septal nuclei, Medial septal nuclei, Lamina terminalis, Vascular organ of lamina terminalis, Olfactory bulb, Piriform cortex, Anterior olfactory nucleus, Olfactory tract, Anterior commissure, Uncus, Cerebral cortex, Frontal lobe, Frontal cortex, Primary motor cortex, Supplementary motor cortex, Premotor cortex, Prefrontal cortex, frontopolar cortex, Orbitofrontal cortex, Dorsolateral prefrontal cortex, dorsomedial prefrontal cortex, ventrolateral prefrontal cortex, Superior frontal gyrus, Middle frontal gyrus, Inferior frontal gyrus, Brodmann areas: 4, 6, 8, 9, 10, 11, 12, 24, 25, 32, 33, 44, 45, 46, 47, Parietal lobe, Parietal cortex, Primary somatosensory cortex (S1), Secondary somatosensory cortex (S2), Posterior parietal cortex, postcentral gyrus, precuneus, Brodmann areas 1, 2, 3 (Primary somesthetic area); 5, 7, 23, 26, 29, 31, 39, 40, Occipital lobe, Primary visual cortex (V1), V2, V3, V4, V5/MT, Lateral occipital gyrus, Cuneus, Brodmann areas 17 (V1, primary visual cortex); 18, 19, temporal lobe, Primary auditory cortex (A1), secondary auditory cortex (A2), Inferior temporal cortex, Posterior inferior temporal cortex, Superior temporal gyrus, Middle temporal gyrus, Inferior temporal gyrus, Entorhinal Cortex, Perirhinal Cortex, Parahippocampal gyrus, Fusiform gyrus, Brodmann areas: 9, 20, 21, 22, 27, 34, 35, 36, 37, 38, 41, 42, Medial superior temporal area (MST), insular cortex, cingulate cortex, Anterior cingulate, Posterior cingulate, dorsal cingulate, Retrosplenial cortex, Indusium griseum, Subgenual area 25, Brodmann areas 23, 24; 26, 29, 30 (retrosplenial areas); 31, 32, cranial nerves (Olfactory (I), Optic (II), Oculomotor (III), Trochlear (IV), Trigeminal (V), Abducens (VI), Facial (VII), Vestibulocochlear (VIII), Glossopharyngeal (IX), Vagus (X), Accessory (XI), Hypoglossal (XII)), and neural pathways Superior longitudinal fasciculus, Arcuate fasciculus, Thalamocortical radiations, Cerebral peduncle, Corpus callosum, Posterior commissure, Pyramidal or corticospinal tract, Medial longitudinal fasciculus, dopamine system, Mesocortical pathway, Mesolimbic pathway, Nigrostriatal pathway, Tuberoinfundibular pathway, serotonin system, Norepinephrine Pathways, Posterior column-medial lemniscus pathway, Spinothalamic tract, Lateral spinothalamic tract, Anterior spinothalamic tract. Brain regions and specific parts of brain regions may be referred to according to their rostral/caudal, dorsal/ventral, medial/lateral, and/or anterior/posterior positions within the brain region with respect to the skull.

The term “brain network”, “brain circuit”, “brain (or neural) connection” or “brain region connectivity” refers to a plurality of brain regions having activity correlated with each other.

The term “brain wave activity” or “oscillatory frequency” as provided herein refers to a repetitive and/or rhythmic neural activity produced by the central nervous system. Brain wave activity or oscillatory frequency can be detected, for example, through the use of an electrode positioned within brain tissue such that the electrode senses voltage fluctuations driven by neural activity. The structure of voltage fluctuations in brain tissue gives rise to oscillatory activity that can be parsed into different frequencies and/or different frequency bands, wherein each frequency band includes a range of frequencies (e.g., delta band including from about 1 Hz to about 4 Hz). “Low frequency” as provided herein refers to brain wave activity including frequencies within a frequency band spanning between 0 Hz to about 38 Hz.

Non-limiting examples of methods for characterizing brain wave activity include power spectral analyses and cross-frequency coupling measures. Power spectral analysis quantifies the power in each frequency or frequency band per unit time. This analysis allows the power in a particular frequency or frequency band (e.g., low frequency) at a given time (e.g., during or immediately prior to manifestation of a disorder symptom) to be compared against the power in the same frequency or frequency band (e.g., low frequency) at a different period in time (e.g., in the absence of a disorder symptom manifestation), thereby allowing detection of power modulations. Alternatively, changes in power in each frequency band may be visually displayed over time by plotting a spectrogram, thereby allowing detection of changes (e.g., modulations) in power in frequencies or frequency bands of interest (e.g., low frequency) to be analyzed over time (e.g., across time periods including or immediately preceding a symptom manifestation, as well as symptom free time periods.).

Cross-frequency coupling measures may be used to describe statistical relationships between frequencies. For example, the phase of low frequency brain wave activity and power of higher frequency (i.e., frequencies faster than those included in low frequency) brain wave activity may have a statistical dependence. Cross-frequency coupling can be assessed at different time points to determine if the statistical dependence of frequencies or frequency bands is modulated by certain conditions (e.g., symptom manifestation).

Brain wave activity may also be related to the activity of individual neurons. A non-limiting example of characterizing the relationship of individual neural activity with brain wave activity is known as spike-field coherence or spike-field coupling. Spike-field coherence quantifies the propensity of action potentials (i.e., spikes) from a given neuron or group of neurons to align with a particular phase of a given frequency of brain wave activity (e.g., low frequency). Spike-field coherence can be assessed at different time points (e.g., periods preceding or concurrent with symptom manifestation and periods temporally distinct from symptom manifestations) such that modulations in spike-field coherence can be determined in response to certain conditions (e.g., symptom manifestation).

As used herein, the term “brain activity level” refers to measurable (e.g., quantifiable) neural activity. Measurable neural activity includes, but is not limited to, a magnitude of activity, a frequency of activity, a delay of activity, or a duration of activity. Brain activity levels may be measured (e.g., quantified) during periods in which no stimulus is presented. In embodiments, the brain activity level measured in the absence of a stimulus is referred to as a baseline brain activity level. Alternatively, brain activity levels may be measured (e.g., quantified) when one or more stimuli are delivered (e.g., an emotional conflict task). In embodiments, the brain activity level measured in the presence of a stimulus is referred to as a brain activity level response. Brain activity levels may be measured simultaneously or sequentially throughout the whole brain, or restricted to specific brain regions (e.g., frontopolar cortex, lateral prefrontal cortex, dorsal anterior cingulate, anterior insula, nucleus accumbens). In embodiments, the brain activity level is determined relative to a baseline brain activity level taken during a baseline period. The baseline period is typically a period during which a stimulus is not presented or has not been presented for a sufficient amount of time (e.g., great than at least 0.05, 0.1, 0.15, 0.25, 0.5, 1, 2, 3, 4, 5, 10, 15, 30, 60 seconds or more). In embodiments, the brain activity level is an oscillatory frequency. In embodiments, the brain activity level is an oscillatory frequency in the ventral striatum. In embodiments, the brain activity level is an oscillatory frequency in the nucleus accumbens.

A brain activity level may also encompass evaluating functional brain region connectivity. For example, neural activity recorded in a plurality of brain regions may have a specific time course across brain regions that can be correlated to reveal a functional brain connectivity pattern (e.g., at a first time point a first brain regions shows an increase in neural activity and at a second time point a second brain region shows an increase in activity). In embodiments, a brain activity level is a measurement (e.g., quantification) of a time course of neural activity across a plurality of brain regions. In embodiments, a brain activity level is a measurement (e.g., quantification) of a time course of neural activity of one brain region (e.g., ventral striatum).

It is contemplated that any suitable method of measuring brain activity levels (e.g., neural activity) including, but not limited to, EEG, MEG, fMRI, and fNIRS may be used for practicing the methods described herein, including embodiments thereof.

In embodiments, the frequency of the brain activity level is detected. In embodiments, the brain activity level detected is a frequency of, for example, delta (0.5-4 Hz), theta (5-8 Hz), alpha (8-12 Hz), beta (12-30 Hz), or gamma (30-60 Hz) or a combination thereof. Similarly, the brain activity level can be measured by detecting the amplitude (e.g., power) of oscillations at delta (0.5-4 Hz), theta (5-8 Hz), alpha (8-12 Hz), beta (12-30 Hz), or gamma (30-60 Hz) frequencies or a combination thereof. In embodiments, the brain activity level frequency, amplitude (e.g., power), and phase can be measured. In embodiments, a duration of the brain activity level is measured. In embodiments, a presence or absence of a brain activity level is measured. In embodiments, a brain activity level may be an average brain activity level. In embodiments, a brain activity level may be a median brain activity level.

In embodiments, a brain activity level is an electrical potential or magnetic field recorded from the nervous system, e.g. brain, of a human or other animal, following presentation of a stimulus (e.g., a trial in an emotional conflict task) that is distinct from spontaneous potentials or fields as detected by electroencephalography (EEG), magnetoencephalography (MEG), or other electrophysiological or neurophysiological recording methods. Such potentials and fields are useful for monitoring brain function in health and disease, and, as described herein, may be used for prognostic purposes. The recorded electrical potential or magnetic field is often presented with an amplitude, phase and/or frequency, including the amplitude or power of the response frequency, which generally indicates an intensity and/or pattern of the response.

As used herein, the term “electroencephalography (EEG)” refers to a non-invasive neurophysiological technique that uses an electronic monitoring device to measure and record electrical activity in the brain.

As used herein, the term “magnetoencephalography (MEG)” refers to a non-invasive neurophysiological technique that measures the magnetic fields generated by neuronal activity of the brain. The spatial distributions of the magnetic fields are analyzed to localize the sources of the activity within the brain.

The term “modulate” is used in accordance with its plain ordinary meaning and refers to the act of changing or varying one or more properties. “Modulation” refers to the process of changing or varying one or more properties (e.g., power, cross-frequency coupling, spike-field-coherence). A modulation may be determined by comparing a test sample to a control sample or value.

A “control” sample or value refers to a sample that serves as a reference or baseline, usually a known reference, for comparison to a test sample. For example, a test sample (e.g., low frequency brain wave activity) can be taken from a patient suffering from a LOC disorder during a time period immediately preceding or concurrent with a disorder symptom manifestation (e.g., binge eating) and compared to a sample from the same patient during a period temporally distinct from a symptom manifestation. A control value can be obtained from the same individual, e.g., from an earlier-obtained sample, prior to disease, or prior to treatment. One of skill will recognize that controls can be designed for assessment of any number of parameters.

It is also contemplated that responses, as measured according to embodiments herein, may be classified (e.g., identified) as, for example, a normal response (e.g., a response similar to a control subject (e.g., healthy control)) or an abnormal response (e.g., a response dissimilar to a control subject or healthy control). This type of classification (e.g., identification) may be useful in, for example, detecting a brain abnormality, diagnosing a brain abnormality, determining a course of treatment, and/or determining treatment outcome. Classification may be carried out by, for example, visual inspect and quantification performed by a human operator. Alternatively, classification may be accomplished via human operator-independent means. For example, classification may be accomplished through a computer running a machine learning model (e.g., algorithm) capable of classifying (e.g., identifying) a response as a normal or abnormal response. The machine learning model may be any suitable machine learning model or algorithm known in the art. In embodiments, the model may be trained, for example using training data, to classify a response as abnormal or normal. Training of the algorithm may be accomplished through supervised or unsupervised training methods.

“Low frequency modulation” as provided herein refers to a change in low frequency brain wave activity (e.g., a change in frequencies between 0 to about 38 Hz) compared to a control. A control may be a baseline low frequency brain wave activity. In embodiments, the baseline low frequency brain wave activity is defined as a time period which is different (longer or shorter (e.g., greater or smaller than 2 seconds)) from the time of manifestation of a disorder symptom. In embodiments, the baseline low frequency brain wave activity is defined as a brain wave frequency different from the frequency characteristic for the manifestation of a disorder symptom. Detection of a low frequency modulation may include methods for characterizing low frequency brain wave activity as described above. Thus, in embodiments, a low frequency modulation is a change in low frequency power relative to a baseline low frequency power. In embodiments, a low frequency modulation is an increase in low frequency power compared to baseline low frequency power. In embodiments, a low frequency modulation is an about 10% to about 45% increase in low frequency power compared to baseline low frequency power. In embodiments, a low frequency modulation is a 10% to 45% increase in low frequency power compared to baseline low frequency power. In embodiments, a low frequency modulation is an about 10% increase in low frequency power compared to baseline low frequency power. In embodiments, a low frequency modulation is a 10% increase in low frequency power compared to baseline low frequency power. In embodiments, a low frequency modulation is an about 15% increase in low frequency power compared to baseline low frequency power. In embodiments, a low frequency modulation is a 15% increase in low frequency power compared to baseline low frequency power. In embodiments, a low frequency modulation is an about 20% increase in low frequency power compared to baseline low frequency power. In embodiments, a low frequency modulation is a 20% increase in low frequency power compared to baseline low frequency power. In embodiments, a low frequency modulation is an about 25% increase in low frequency power compared to baseline low frequency power. In embodiments, a low frequency modulation is a 25% increase in low frequency power compared to baseline low frequency power. In embodiments, a low frequency modulation is an about 30% increase in low frequency power compared to baseline low frequency power. In embodiments, a low frequency modulation is a 30% increase in low frequency power compared to baseline low frequency power. In embodiments, a low frequency modulation is an about 35% increase in low frequency power compared to baseline low frequency power. In embodiments, a low frequency modulation is a 35% increase in low frequency power compared to baseline low frequency power. In embodiments, a low frequency modulation is an about 40% increase in low frequency power compared to baseline low frequency power. In embodiments, a low frequency modulation is a 40% increase in low frequency power compared to baseline low frequency power. In embodiments, a low frequency modulation is an about 45% increase in low frequency power compared to baseline low frequency power. In embodiments, a low frequency modulation is a 45% increase in low frequency power compared to baseline low frequency power.

In embodiments, a low frequency modulation includes a modulation in cross-frequency coupling between low frequency brain wave activity and higher frequency brain wave activity.

In embodiments, a low frequency modulation is a modulation in low frequency spike-field coherence. In embodiments, a low frequency modulation is an increase in low frequency spike-field coherence.

In embodiments, a low frequency modulation precedes the onset of a disorder symptom manifestation. In embodiments, a low frequency modulation precedes the onset of a disorder symptom manifestation by about 2 seconds. In embodiments, a low frequency modulation precedes the onset of a disorder symptom manifestation by 2 seconds. In embodiments, a low frequency modulation precedes the onset of a disorder symptom manifestation by about 1.5 seconds. In embodiments, a low frequency modulation precedes the onset of a disorder symptom manifestation by 1.5 seconds. In embodiments, a low frequency modulation precedes the onset of a disorder symptom manifestation by about 1 second. In embodiments, a low frequency modulation precedes the onset of a disorder symptom manifestation by 1 second. In embodiments, a low frequency modulation precedes the onset of a disorder symptom manifestation by about 0.5 seconds. In embodiments, a low frequency modulation precedes the onset of a disorder symptom manifestation by 0.5 seconds. In embodiments, a low frequency modulation precedes the onset of a disorder symptom manifestation by about 0.1 seconds. In embodiments, a low frequency modulation precedes the onset of a disorder symptom manifestation by 0.1 seconds. Thus, the low frequency modulation is predictive of a disease symptom manifestation (e.g., binge eating). In embodiments, the low frequency modulation is a biomarker.

A “biomarker” as provided herein refers to any assayable characteristics or compositions that are used to identify, predict, or monitor a condition (e.g., symptom of an OCD disorder), a therapy for said condition in a subject or sample or the location of an anatomic structure in the brain. A biomarker is, for example, an oscillatory frequency (e.g., 35 Hz) whose presence is used to identify the anatomic structure (anatomic location in the brain) of the Nucleus accumbens in a subject. Biomarkers identified herein are measured to determine the anatomic structure (location in the brain), the onset of disease symptoms and to serve as a trigger for delivering (e.g., administering) a therapeutic stimulation (i.e., electrical stimulation).

The term “electrical stimulation” as used herein refers to an electromagnetic energy administered to the brain in a precise location using an electrode, wherein said electromagnetic energy is capable of modulating an electrical impulse in the brain (e.g., reducing low frequency power in brain region). The electromagnetic energy may be administered at specific parameters which include, for example, frequency, time (burst duration), duty cycle and repetition or any combination thereof. The term “burst duration” as used herein refers to the length of time during which the electrical impulses at a given frequency are administered. Likewise, a “burst” as referred to herein corresponds to the electrical impulse administered at a given frequency. A “duty cycle” as used herein refers to the number and sequence of burst durations (e.g., time-on) followed by the time wherein no burst is administered (e.g., time-off).

The terms “dose” and “dosage” are used interchangeably herein and are defined by the specific parameters of administering an electrical stimulation. Therefore, a dose as provided herein refers to an electrical stimulus administered at a given frequency, burst duration, duty cycle, repetition or any combination thereof. The dose will vary depending on a number of factors, including the range of normal doses for a given therapy; frequency of administration; size and tolerance of the individual; severity of the condition; and risk of side effects. One of skill will recognize that the dose can be modified depending on the above factors or based on therapeutic progress. In the present invention, the dose may undergo multiple iterations in order to optimize a therapeutic effect.

As used herein, the terms “treat,” treating,” “treatment,” and the like refer to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” and “treatment” as used herein include prophylactic treatment. Treatment methods include administering to a subject a therapeutically effective amount of an active agent (i.e., electrical stimulation). The administering step may consist of a single administration or may include a series of administrations. The length of the treatment period depends on a variety of factors, such as the severity of the condition, the age of the patient, the concentration of active agent (e.g., electrical stimulation), the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, electrical stimulations are administered to the subject in an amount and for a duration sufficient to treat the patient.

The term “prevent” refers to a decrease in the occurrence of LOC-associated disorder symptoms in a patient. As indicated above, the prevention may be complete (no detectable symptoms) or partial, such that fewer symptoms are observed than would likely occur absent treatment.

A “subject” as used herein refers to an organism. In embodiments, the organism is an animal. In embodiments, the subject is a living organism. In embodiments, the subject is a cadaver organism. In embodiments, the subject is a mammal, including, but not limited to, a human or non-human mammal. In embodiments, the subject is a domesticated mammal or a primate including a non-human primate. Examples of subjects include humans, monkeys, dogs, cats, mice, rats, cows, horses, goats, and sheep. A human subject may also be referred to as a patient. In embodiments, the subject suffers from a neurological disorder. In embodiments, the subject suffers from a psychiatric disorder. In embodiments, the subject suffers from OCD.

“Control subject”, “healthy control”, “normal non-diseased control subject”, or “standard control”, or the like (e.g., control population), is used in accordance with its plain ordinary meaning and refers herein to a subject not suffering from a disease, condition, syndrome, abnormality, or disorder (e.g., OCD, brain dysfunction, brain abnormality, brain disorder). In instances, the control subject is used as a standard of comparison in evaluating experimental effects. In some instances, the control subject is used as a standard of comparison in the process of diagnosing or prognosing a subject suffering from, suspected of suffering from, not suspected of suffering from, or not clinically diagnosed with a disease, condition, syndrome, abnormality, or disorder (e.g., brain dysfunction, brain abnormality, brain disorder).

A subject “suffering from or suspected of suffering from” a specific disease, disorder, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from conditions associated with OCD is within the ability of those in the art. Subjects suffering from, and suspected of suffering from, a specific disease, disorder, condition, or syndrome are not necessarily two distinct groups.

As used herein, “susceptible to” or “prone to” or “predisposed to” a specific disease or condition and the like refers to an individual who based on genetic, environmental, health, and/or other risk factors is more likely to develop a disease, disorder, or condition than the general population. An increase in likelihood of developing a disease may be an increase of about 10%, 20%, 50%, 100%, 150%, 200%, or more.

The term “therapeutically effective amount,” as used herein, refers to the amount or dose of a therapeutic agent (i.e., electrical stimulation) sufficient to ameliorate the disorder, as described above. For example, for the given dose, a therapeutically effective amount will show an increase of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

The term “administering” as provided herein, refers to the delivery of an electrical stimulation via one or more electrodes positioned within a specific brain structure (e.g., NAc). In the present invention, administration may be commenced following detection of a biomarker (e.g., an oscillatory frequency, e.g., of 35 Hz). In embodiments, administration is accomplished by the apparatus and system provided herein, including embodiments thereof. The same device used to administer electrical stimulation can be used to record brain wave activity to detect a biomarker of an anatomic structure (e.g., Nac) or a marker of a disease. In embodiments, administration is triggered automatically by detection of a biomarker of an anatomic structure (e.g., Nac) or a marker of a disease. This method of biomarker detection may be followed by automatic electrical stimulation administration referred to herein as “closed-loop” neurostimulation or responsive neurostimulation (RNS). This form of stimulation differs from deep brain stimulation (DBS) in that deep brain stimulation is not a closed-loop system, but rather sends chronic and continuous electrical impulses through the implanted electrodes to specific brain targets. Thus, DBS may be referred to herein as an “open-loop” type of therapeutic treatment, because it involves continuous electrical stimulation that is not preceded by detection of or triggered by specific biomarkers. Where a dose provided herein is compared to a dose administered in DBS, the dose is generally compared to a dose in an open-loop type system.

The terms “aberrant”, “abnormal”, “impairment”, and the like, as used herein refer to different from normal. When used to describe, for example, responses (e.g., variables as described herein) or brain circuit function, abnormal refers to responses (e.g., variable as described herein) or brain circuit function that is greater or less than a normal non-diseased control subject, the average of normal non-diseased control subjects, wherein an average may be the mean or median of a control population, or a specific amount (e.g., 1.5 standard deviations) outside of the distribution of responses determined in a population of normal non-diseased control subjects. An abnormal response may refer to an amount of activity (e.g., over or under activity (e.g., neural activity), variable as described herein) that results in or is indicative of a disease, wherein returning the abnormal response to a normal response or non-disease-associated response as may be observed in a control subject (e.g. by administering a non-invasive stimulation treatment), results in reduction of the disease or one or more disease symptoms. In embodiments, an abnormal response or impaired response is at least 5%, 10%, 15%, 20%, 30%, 40%, 50%, 60%, 70%, 80%, 90%, 100% or more different (e.g., positively or negatively) from a response measured in a control subject. In embodiments, an abnormal response is a response that falls 1.5 standard deviations or more from the response distribution measured in a control population. In embodiments, an abnormal response or impaired response is at least 5%, 10%, 15%, 20%, 30%, 40%, 50%, 60%, 70%, 80%, 90%, 100% or more different (e.g., positively or negatively) from the average response measured in a control population. In embodiments, an abnormal response or impaired response is at least 5%, 10%, 15%, 20%, 30%, 40%, 50%, 60%, 70%, 80%, 90%, 100% or more different (e.g., positively or negatively) from the median response measured in a control population.

The abnormal response may be identified by comparing one or a combination of variables as described herein in a subject against one or a combination of variables as described herein in a control subject or population. In embodiments, a machine learning model is used to identify an abnormal response.

A “reduction” of a symptom or symptoms (and grammatical equivalents of this phrase) means a decreasing of the severity or frequency of the symptom(s), or elimination of the symptom(s) (e.g. in response to a treatment relative to the absence of the treatment).

The term “therapeutically effective amount,” as used herein, refers to that amount of the therapeutic agent (i.e., electrical stimulation) sufficient to ameliorate or reduce symptoms of the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the non-invasive stimulation being employed. The dose administered to a patient, in the context of the present disclosure, should be sufficient to effect a beneficial therapeutic response in the patient over time. The structure of the dose (e.g., frequency and amount of electrical stimulation) also will be determined by the existence, nature, and extent of any adverse side-effects. Determination of the proper dosage for a particular situation is within the skill of the practitioner. Generally, treatment is initiated with smaller dosages which are less than the optimum dose of the non-invasive stimulation. Thereafter, the dosage is increased by small increments until the optimum effect under circumstances is reached. Dosage amounts (e.g., intensity) and intervals (e.g., treatment sessions) can be adjusted individually to provide levels of the administered stimulation effective for the particular clinical indication being treated. This will provide a therapeutic regimen that is commensurate with the severity of the individual's disease state.

As used herein, the term “administering” refers to non-invasive or invasive brain stimulation administration. In embodiments, the administering does not include administration of any active agent other than the non-invasive stimulation. In embodiments, the administering includes administration of one or more active agents (e.g., medications) in addition to the non-invasive stimulation. In embodiments, the administering does not include administration of any active agent other than the invasive stimulation. In embodiments, the administering includes administration of one or more active agents (e.g., medications) in addition to the invasive stimulation.

### Methods

Provided herein are, inter alia, methods for detecting the anatomic structure of a nucleus accumbens in the brain of a subject. The method includes inserting an electrode into the ventral striatum of a subject; and detecting an oscillatory frequency of 30-40 Hz, thereby identifying the anatomic structure of a nucleus accumbens in the subject. The methods provided may include a step of delivering an electrical stimulation to ameliorate or prevent an OCD symptom from occurring. Thus, in one aspect a method of identifying the anatomic structure of a nucleus accumbens in a subject is provided, the method including inserting an electrode into the ventral striatum of a subject, and detecting an oscillatory frequency of 30-40 Hz, thereby identifying the anatomic structure of a nucleus accumbens in the subject.

In embodiments, the oscillatory frequency is a frequency of 26-44 Hz. In embodiments, the oscillatory frequency is a frequency of 27-44 Hz. In embodiments, the oscillatory frequency is a frequency of 28-44 Hz. In embodiments, the oscillatory frequency is a frequency of 29-44 Hz. In embodiments, the oscillatory frequency is a frequency of 30-44 Hz. In embodiments, the oscillatory frequency is a frequency of 31-44 Hz. In embodiments, the oscillatory frequency is a frequency of 32-44 Hz. In embodiments, the oscillatory frequency is a frequency of 33-44 Hz. In embodiments, the oscillatory frequency is a frequency of 34-44 Hz. In embodiments, the oscillatory frequency is a frequency of 35-44 Hz. In embodiments, the oscillatory frequency is a frequency of 36-44 Hz. In embodiments, the oscillatory frequency is a frequency of 37-44 Hz. In embodiments, the oscillatory frequency is a frequency of 38-44 Hz. In embodiments, the oscillatory frequency is a frequency of 39-44 Hz. In embodiments, the oscillatory frequency is a frequency of 40-44 Hz. In embodiments, the oscillatory frequency is a frequency of 41-44 Hz. In embodiments, the oscillatory frequency is a frequency of 42-44 Hz.

In embodiments, the oscillatory frequency is a frequency of 26-42 Hz. In embodiments, the oscillatory frequency is a frequency of 26-41 Hz. In embodiments, the oscillatory frequency is a frequency of 26-40 Hz. In embodiments, the oscillatory frequency is a frequency of 26-39 Hz. In embodiments, the oscillatory frequency is a frequency of 26-38 Hz. In embodiments, the oscillatory frequency is a frequency of 26-37 Hz. In embodiments, the oscillatory frequency is a frequency of 26-38 Hz. In embodiments, the oscillatory frequency is a frequency of 26-36 Hz. In embodiments, the oscillatory frequency is a frequency of 26-35 Hz. In embodiments, the oscillatory frequency is a frequency of 26-34 Hz. In embodiments, the oscillatory frequency is a frequency of 26-33 Hz. In embodiments, the oscillatory frequency is a frequency of 26-32 Hz. In embodiments, the oscillatory frequency is a frequency of 26-31 Hz. In embodiments, the oscillatory frequency is a frequency of 26-30 Hz. In embodiments, the oscillatory frequency is a frequency of 26-29 Hz. In embodiments, the oscillatory frequency is a frequency of 26-28 Hz. In embodiments, the oscillatory frequency is a frequency of 44 Hz, 43 Hz, 42 Hz, 41 Hz, 40 Hz, 39 Hz, 38 Hz, 37 Hz, 36 Hz, 35 Hz, 34 Hz, 32 Hz, 31 Hz, 30 Hz, 29 Hz, 28 Hz, 27 Hz, or 26 Hz. In embodiments, the oscillatory frequency is a frequency of about 35 Hz. In embodiments, the oscillatory frequency is a frequency of 35 Hz.

In embodiments the electrode is a microelectrode. In embodiments, the method further includes detecting a theta oscillatory frequency or an alpha oscillatory frequency. In embodiments, the method further includes detecting a theta oscillatory frequency. In embodiments, the method further includes detecting an alpha oscillatory frequency. In embodiments, the method further includes detecting a theta oscillatory frequency and an alpha oscillatory frequency.

In embodiments, the subject is suffering from obsessive compulsive disorder.

In embodiments, including after the detecting administering an electrical stimulation to the nucleus accumbens of the subject. In embodiments, a dose of electrical stimulation is less than a dose corresponding to deep brain stimulation.

In embodiments, the electrical stimulation is administered at a frequency of 2-16 Hz. In embodiments, the electrical stimulation is administered at a frequency of 3-16 Hz. In embodiments, the electrical stimulation is administered at a frequency of 4-16 Hz. In embodiments, the electrical stimulation is administered at a frequency of 5-16 Hz. In embodiments, the electrical stimulation is administered at a frequency of 6-16 Hz. In embodiments, the electrical stimulation is administered at a frequency of 7-16 Hz. In embodiments, the electrical stimulation is administered at a frequency of 8-16 Hz. In embodiments, the electrical stimulation is administered at a frequency of 9-16 Hz. In embodiments, the electrical stimulation is administered at a frequency of 10-16 Hz. In embodiments, the electrical stimulation is administered at a frequency of 11-16 Hz. In embodiments, the electrical stimulation is administered at a frequency of 12-16 Hz. In embodiments, the electrical stimulation is administered at a frequency of 13-16 Hz. In embodiments, the electrical stimulation is administered at a frequency of 14-16 Hz.

In embodiments, the electrical stimulation is administered at a frequency of 2-14 Hz. In embodiments, the electrical stimulation is administered at a frequency of 2-13 Hz. In embodiments, the electrical stimulation is administered at a frequency of 2-12 Hz. In embodiments, the electrical stimulation is administered at a frequency of 2-11 Hz. In embodiments, the electrical stimulation is administered at a frequency of 2-10 Hz. In embodiments, the electrical stimulation is administered at a frequency of 2-9 Hz. In embodiments, the electrical stimulation is administered at a frequency of 2-8 Hz. In embodiments, the electrical stimulation is administered at a frequency of 2-7 Hz. In embodiments, the electrical stimulation is administered at a frequency of 2-6 Hz. In embodiments, the electrical stimulation is administered at a frequency of 2-5 Hz. In embodiments, the electrical stimulation is administered at a frequency of 2-4 Hz. In embodiments, the electrical stimulation is administered at a frequency of 16 Hz, 15 Hz, 14 Hz, 13 Hz, 12 Hz, 11 Hz, 10 Hz, 9 Hz, 8 Hz, 7 Hz, 6 Hz, 5 Hz, 4 Hz, 3 Hz, or 2 Hz.

In embodiments, the electrical stimulation is administered at a frequency of about 5 Hz. In embodiments, the electrical stimulation is administered at a frequency of 5 Hz. In embodiments, the electrical stimulation is administered at a frequency of about 10 Hz. In embodiments, the electrical stimulation is administered at a frequency of 10 Hz. In embodiments, the electrical stimulation is administered at a frequency of about 12 Hz. In embodiments, the electrical stimulation is administered at a frequency of 12 Hz.

In embodiments, the electrical stimulation is administered at a frequency of 5 Hz, 10 Hz, 12 Hz, 160 Hz, 212 Hz, or 333 Hz In embodiments, the electrical stimulation is administered at a frequency of 140-180 Hz. In embodiments, the electrical stimulation is administered at a frequency of 145-180 Hz. In embodiments, the electrical stimulation is administered at a frequency of 150-180 Hz. In embodiments, the electrical stimulation is administered at a frequency of 155-180 Hz. In embodiments, the electrical stimulation is administered at a frequency of 160-180 Hz. In embodiments, the electrical stimulation is administered at a frequency of 165-180 Hz. In embodiments, the electrical stimulation is administered at a frequency of 170-180 Hz. In embodiments, the electrical stimulation is administered at a frequency of 175-180 Hz. In embodiments, the electrical stimulation is administered at a frequency of 140-175 Hz. In embodiments, the electrical stimulation is administered at a frequency of 140-170 Hz. In embodiments, the electrical stimulation is administered at a frequency of 140-165 Hz. In embodiments, the electrical stimulation is administered at a frequency of 140-160 Hz. In embodiments, the electrical stimulation is administered at a frequency of 140-155 Hz. In embodiments, the electrical stimulation is administered at a frequency of 140-150 Hz. In embodiments, the electrical stimulation is administered at a frequency of 140-145 Hz. In embodiments, the electrical stimulation is administered at a frequency of 180 Hz, 175 Hz, 170 Hz, 165 Hz, 160 Hz, 155 Hz, 150 Hz, 145 Hz or 140 Hz.

In embodiments, the electrical stimulation is administered at a frequency of about 160 Hz. In embodiments, the electrical stimulation is administered at a frequency of 160 Hz.

In embodiments, the electrical stimulation is administered at a frequency of 204-220 Hz. In embodiments, the electrical stimulation is administered at a frequency of 206-220 Hz. In embodiments, the electrical stimulation is administered at a frequency of 208-220 Hz. In embodiments, the electrical stimulation is administered at a frequency of 210-220 Hz. In embodiments, the electrical stimulation is administered at a frequency of 212-220 Hz. In embodiments, the electrical stimulation is administered at a frequency of 214-220 Hz. In embodiments, the electrical stimulation is administered at a frequency of 216-220 Hz. In embodiments, the electrical stimulation is administered at a frequency of 218-220 Hz.

In embodiments, the electrical stimulation is administered at a frequency of 204-218 Hz. In embodiments, the electrical stimulation is administered at a frequency of 204-216 Hz. In embodiments, the electrical stimulation is administered at a frequency of 204-214 Hz. In embodiments, the electrical stimulation is administered at a frequency of 204-212 Hz. In embodiments, the electrical stimulation is administered at a frequency of 204-210 Hz. In embodiments, the electrical stimulation is administered at a frequency of 204-208 Hz. In embodiments, the electrical stimulation is administered at a frequency of 204-206 Hz. In embodiments, the electrical stimulation is administered at a frequency of 220 Hz, 218 Hz, 216 Hz, 214 Hz, 212 Hz, 210 Hz, 208 Hz, 206 Hz or 204 Hz.

In embodiments, the electrical stimulation is administered at a frequency of about 212 Hz. In embodiments, the electrical stimulation is administered at a frequency of 212 Hz

In embodiments, the electrical stimulation is administered at a frequency of 327-339 Hz. In embodiments, the electrical stimulation is administered at a frequency of 330-339 Hz. In embodiments, the electrical stimulation is administered at a frequency of 333-339 Hz. In embodiments, the electrical stimulation is administered at a frequency of 336-339 Hz. In embodiments, the electrical stimulation is administered at a frequency of 327-336 Hz. In embodiments, the electrical stimulation is administered at a frequency of 327-333 Hz. In embodiments, the electrical stimulation is administered at a frequency of 327-330 Hz. In embodiments, the electrical stimulation is administered at a frequency of 339 Hz, 336 Hz, 333 Hz, 330 Hz or 327 Hz.

In embodiments, the electrical stimulation is administered at a frequency of about 333 Hz. In embodiments, the electrical stimulation is administered at a frequency of 333 Hz

In embodiments, the electrical stimulation is administered at 100 milliseconds, 1 minute, 15 minutes, or 1 hour.

In embodiments, the electrical stimulation is administered at 80-120 milliseconds. In embodiments, the electrical stimulation is administered at 85-120 milliseconds. In embodiments, the electrical stimulation is administered at 90-120 milliseconds. In embodiments, the electrical stimulation is administered at 95-120 milliseconds. In embodiments, the electrical stimulation is administered at 100-120 milliseconds. In embodiments, the electrical stimulation is administered at 105-120 milliseconds. In embodiments, the electrical stimulation is administered at 110-120 milliseconds. In embodiments, the electrical stimulation is administered at 115-120 milliseconds. In embodiments, the electrical stimulation is administered at 80-115 milliseconds. In embodiments, the electrical stimulation is administered at 80-110 milliseconds. In embodiments, the electrical stimulation is administered at 80-105 milliseconds. In embodiments, the electrical stimulation is administered at 80-100 milliseconds. In embodiments, the electrical stimulation is administered at 80-95 milliseconds. In embodiments, the electrical stimulation is administered at 80-90 milliseconds. In embodiments, the electrical stimulation is administered at 80-85 milliseconds. In embodiments, the electrical stimulation is administered at 120, 115, 110, 100, 95, 90, 95 or 80 milliseconds.

In embodiments, the electrical stimulation is administered at about 100 milliseconds. In embodiments, the electrical stimulation is administered at 100 milliseconds.

In embodiments, the electrical stimulation is administered at 0.2-1.8 minutes. In embodiments, the electrical stimulation is administered at 0.4-1.8 minutes. In embodiments, the electrical stimulation is administered at 0.6-1.8 minutes. In embodiments, the electrical stimulation is administered at 0.8-1.8 minutes. In embodiments, the electrical stimulation is administered at 1.0-1.8 minutes. In embodiments, the electrical stimulation is administered at 1.2-1.8 minutes. In embodiments, the electrical stimulation is administered at 1.4-1.8 minutes. In embodiments, the electrical stimulation is administered at 1.6-1.8 minutes. In embodiments, the electrical stimulation is administered at 0.2-1.6 minutes. In embodiments, the electrical stimulation is administered at 0.2-1.4 minutes. In embodiments, the electrical stimulation is administered at 0.2-1.2 minutes. In embodiments, the electrical stimulation is administered at 0.2-1.0 minutes. In embodiments, the electrical stimulation is administered at 0.2-0.8 minutes. In embodiments, the electrical stimulation is administered at 0.2-0.6 minutes. In embodiments, the electrical stimulation is administered at 0.2-0.4 minutes. In embodiments, the electrical stimulation is administered at 1.8, 1.6, 1.4, 1.2, 1.0, 0.8, 0.6, 0.4 or 0.2 minutes.

In embodiments, the electrical stimulation is administered at about 1 minute. In embodiments, the electrical stimulation is administered at 1 minute.

In embodiments, the electrical stimulation is administered at 10-20 minutes. In embodiments, the electrical stimulation is administered at 12-20 minutes. In embodiments, the electrical stimulation is administered at 14-20 minutes. In embodiments, the electrical stimulation is administered at 16-20 minutes. In embodiments, the electrical stimulation is administered at 18-20 minutes. In embodiments, the electrical stimulation is administered at 10-18 minutes. In embodiments, the electrical stimulation is administered at 10-16 minutes. In embodiments, the electrical stimulation is administered at 10-14 minutes. In embodiments, the electrical stimulation is administered at 10-12 minutes. In embodiments, the electrical stimulation is administered at 20, 18, 16, 14, 12 or 10 minutes.

In embodiments, the electrical stimulation is administered at about 15 minutes. In embodiments, the electrical stimulation is administered at 15 minutes.

In embodiments, the electrical stimulation is administered at 0.5 to 1.5 hours. In embodiments, the electrical stimulation is administered at 0.6 to 1.5 hours. In embodiments, the electrical stimulation is administered at 0.7 to 1.5 hours. In embodiments, the electrical stimulation is administered at 0.8 to 1.5 hours. In embodiments, the electrical stimulation is administered at 0.9 to 1.5 hours. In embodiments, the electrical stimulation is administered at 1 to 1.5 hours. In embodiments, the electrical stimulation is administered at 1.1 to 1.5 hours. In embodiments, the electrical stimulation is administered at 1.2 to 1.5 hours. In embodiments, the electrical stimulation is administered at 1.3 to 1.5 hours. In embodiments, the electrical stimulation is administered at 1.4 to 1.5 hours. In embodiments, the electrical stimulation is administered at 0.5-1.4 hours. In embodiments, the electrical stimulation is administered at 0.5-1.3 hours. In embodiments, the electrical stimulation is administered at 0.5-1.2 hours. In embodiments, the electrical stimulation is administered at 0.5-1.1 hours. In embodiments, the electrical stimulation is administered at 0.5-1 hours. In embodiments, the electrical stimulation is administered at 0.5-0.9 hours. In embodiments, the electrical stimulation is administered at 0.5-0.8 hours. In embodiments, the electrical stimulation is administered at 0.5-0.7 hours. In embodiments, the electrical stimulation is administered at 0.5-0.6 hours. In embodiments, the electrical stimulation is administered at 1.5, 1.4, 1.3, 1.2, 1.1, 1.0, 0.9, 0.6, 0.7, 0.6 or 0.5 hours.

In embodiments, the electrical stimulation is administered at about 1 hour. In embodiments, the electrical stimulation is administered 1 hour.

In an aspect a method of treating an obsessive compulsive disorder (OCD) subject is provided, the method including detecting an oscillatory frequency of 30-40 Hz in the ventral striatum of an OCD subject, and administering an electrical stimulation to the nucleus accumbens of the OCD subject in response to the oscillatory frequency, thereby treating the OCD subject.

In embodiments, the oscillatory frequency is a frequency of about 35 Hz. In embodiments, the method further includes detecting a theta oscillatory frequency or an alpha oscillatory frequency. In embodiments, a dose of electrical stimulation is less than a dose corresponding to deep brain stimulation. In embodiments, the electrical stimulation is administered at a frequency of 5 Hz, 10 Hz, 12 Hz, 160 Hz, 212 Hz or 333 Hz. In embodiments, the electrical stimulation is administered at 100 milliseconds, 1 minute, 15 minutes, or 1 hour. Any of the doses, frequencies and stimulation times described herein are contemplated for the methods of treatment provided herewith. For example, the oscillatory frequency may be a frequency of 26-42 Hz, 26-41 Hz, 26-40 Hz, 26-35 Hz; the electrical stimulation may be administered at a frequency of 2-16 Hz, 3-16 Hz, 4-16 Hz, 5-16 Hz, 6-16 Hz, 155-180 Hz, 160-180 Hz, 165-180 Hz; the electrical stimulation may be administered at 80-120 milliseconds, 85-120 milliseconds, 90-120 milliseconds, 95-120 milliseconds, or 100-120 milliseconds.

In embodiments, at least 2 electrodes are inserted into the nucleus accumbens. In embodiments, at least 3 electrodes are inserted into the nucleus accumbens. In embodiments, at least 4 electrodes are inserted into the nucleus accumbens. In embodiments, at least 5 electrodes are inserted into the nucleus accumbens. In embodiments, at least 6 electrodes are inserted into the nucleus accumbens. In embodiments, at least 7 electrodes are inserted into the nucleus accumbens. In embodiments, at least 8 electrodes are inserted into the nucleus accumbens.

In embodiments, 2 electrodes are inserted into the nucleus accumbens. In embodiments, 3 electrodes are inserted into the nucleus accumbens. In embodiments, 4 electrodes are inserted into the nucleus accumbens. In embodiments, 5 electrodes are inserted into the nucleus accumbens. In embodiments, 6 electrodes are inserted into the nucleus accumbens. In embodiments, 7 electrodes are inserted into the nucleus accumbens. In embodiments, 8 electrodes are inserted into the nucleus accumbens. In embodiments, at least one of the electrodes inserted is a deep brain electrode.

In embodiments, electrodes are inserted unilaterally into a nucleus accumbens of the subject. In embodiments, electrodes are inserted bilaterally into the nucleus accumbens of the subject.

In embodiments, the at least one electrode is a deep brain electrode. A deep brain electrode as used herein refers to an electrode capable of targeting a deep brain structure (e.g., NAc).

In the present invention, detection of a biomarker (oscillatory frequency of 30-40 Hz) of an anatomic structure (e.g., Nac) may be combined with the administration of an electrical stimulus to ameliorate or prevent manifestation of an OCD symptom. Therefore, in embodiments, the method further includes administering, in response to detecting an oscillatory frequency of 30-40 Hz, an electrical stimulation to the nucleus accumbens of the subject.

Both the detecting the oscillatory frequency of 30-40 Hz (e.g., 35 Hz) and delivery of the electrical stimulation may occur via the one or more electrodes positioned within the nucleus accumbens. In embodiments, an electrode records brain wave activity and delivers an electrical stimulation. In embodiments, the electrode recording brain wave activity and the electrode delivering an electrical stimulation are the same. In embodiments, the brain wave activity is recorded a first electrode and an electrical stimulation is delivered by the first electrode. In embodiments, a subset of electrodes (e.g., one or more additional electrodes) record the oscillatory frequency of 30-40 Hz and a different subset of electrodes deliver electrical stimulation. In embodiments, a first electrode records the oscillatory frequency of 30-40 Hz and a second electrode delivers electrical stimulation. In embodiments, the first electrode and the second electrode are the same. In embodiments, the first electrode and the second electrode are different. In embodiments, a plurality (i.e. more than one) of first electrodes records the oscillatory frequency of 30-40 Hz and a plurality (i.e. more than one) of second electrodes delivers electrical stimulation. In embodiments, the plurality of first electrodes is the same or different. In embodiments, the plurality of second electrodes is the same or different. In embodiments, the plurality of first electrodes and the plurality of second electrodes are different. In embodiments, the plurality of first electrodes and the plurality of second electrodes are the same. In embodiments, a first electrode records the oscillatory frequency of 30-40 Hz and a plurality (i.e. more than one) of second electrodes delivers electrical stimulation. In embodiments, a plurality (i.e. more than one) of first electrodes records the oscillatory frequency of 30-40 Hz and a second electrode delivers electrical stimulation.

In embodiments, a subset of electrodes record the oscillatory frequency of 30-40 Hz and deliver electrical stimulation and a different subset of electrodes record the oscillatory frequency of 30-40 Hz. In embodiments, a plurality of first electrodes record the oscillatory frequency of 30-40 Hz and deliver electrical stimulation and a plurality of second electrodes record the oscillatory frequency of 30-40 Hz. In embodiments, a subset of electrodes record the oscillatory frequency of 30-40 Hz and deliver electrical stimulation and a different subset of electrodes deliver electrical stimulation. In embodiments, a plurality of first electrodes record the oscillatory frequency of 30-40 Hz and deliver electrical stimulation and a plurality of second electrodes deliver electrical stimulation. It will be obvious to one skilled in the art that numerous electrode configurations may be used to detect (record) oscillatory frequency of 30-40 Hz and deliver electrical stimulations.

The electrical stimulation (e.g., dosage) administered can vary in frequency, burst duration, duty cycle, repetition, etc. In embodiments, a dose of the electrical stimulation is less than a dose corresponding to deep brain stimulation. This may occur, for example if the electrical stimulation is not administered continuously or in an open-loop configuration.

In embodiments, electric stimulation is applied at 5 hertz (Hz), 10 hertz (Hz), 12 hertz (Hz), 160 hertz (Hz), 212 hertz (Hz), or 333 hertz (Hz). In embodiments, electric stimulation is applied at 5 hertz. In embodiments, electric stimulation is applied at 10 hertz. In embodiments, electric stimulation is applied at 12 hertz. In embodiments, electric stimulation is applied at 160 hertz. In embodiments, electric stimulation is applied at 212 hertz. In embodiments, electric stimulation is applied at 333 hertz. In embodiments, electric stimulation is applied at 130 hertz.

In embodiments, bursts of electrical stimulation are applied with a burst duration of the electrical stimulation being 100 milliseconds, 1 minute, 15 minutes, or 1 hour. In embodiments, bursts of electrical stimulation are applied with a burst duration of the electrical stimulation being 100 milliseconds. In embodiments, bursts of electrical stimulation are applied with a burst duration of the electrical stimulation being 1 minute. In embodiments, bursts of electrical stimulation are applied with a burst duration of the electrical stimulation being 15 minutes. In embodiments, bursts of electrical stimulation are applied with a burst duration of the electrical stimulation being 1 hour.

In embodiments, a duty cycle of the electrical stimulation is continuous, bursting, or on for a length of time and off for a different length of time. In embodiments, a duty cycle of the electrical stimulation is continuous. In embodiments, a duty cycle of the electrical stimulation is bursting. In embodiments, a duty cycle of the electrical stimulation is on for a length of time and off for a different length of time.

In embodiments, the low frequency modulation includes a modulation having a frequency between about 0 hertz-38 hertz. In embodiments, the low frequency modulation includes a modulation having a frequency between 0 hertz-38 hertz. In embodiments, the low frequency modulation includes a modulation having a frequency between about 0 hertz-30 hertz. In embodiments, the low frequency modulation includes a modulation having a frequency between 0 hertz-30 hertz. In embodiments, the low frequency modulation includes a modulation having a frequency between about 0 hertz-25 hertz. In embodiments, the low frequency modulation includes a modulation having a frequency between 0 hertz-25 hertz. In embodiments, the low frequency modulation includes a modulation having a frequency between about 0 hertz-20 hertz. In embodiments, the low frequency modulation includes a modulation having a frequency between 0 hertz-20 hertz. In embodiments, the low frequency modulation includes a modulation having a frequency between about 0 hertz-15 hertz. In embodiments, the low frequency modulation includes a modulation having a frequency between 0 hertz-15 hertz. In embodiments, the low frequency modulation includes a modulation having a frequency between about 0 hertz-12 hertz. In embodiments, the low frequency modulation includes a modulation having a frequency between 0 hertz-12 hertz. In embodiments, the low frequency modulation includes a modulation having a frequency between about 0 hertz-10 hertz. In embodiments, the low frequency modulation includes a modulation having a frequency between 0 hertz-10 hertz. In embodiments, the low frequency modulation includes a modulation having a frequency between about 0 hertz-8 hertz. In embodiments, the low frequency modulation includes a modulation having a frequency between 0 hertz-8 hertz. In embodiments, the low frequency modulation includes a modulation having a frequency between about 0 hertz-4 hertz. In embodiments, the low frequency modulation includes a modulation having a frequency between 0 hertz-4 hertz. In embodiments, the low frequency modulation includes a modulation having a frequency between about 0 hertz-3 hertz. In embodiments, the low frequency modulation includes a modulation having a frequency between 0 hertz-3 hertz. In embodiments, the low frequency modulation includes a modulation having a frequency between about 1 hertz-4 hertz. In embodiments, the low frequency modulation includes a modulation having a frequency between 1 hertz-4 hertz.

Implementations of the present disclosure can include, but are not limited to, methods consistent with the descriptions provided above as well as articles that comprise a tangibly embodied machine-readable medium operable to cause one or more machines (e.g., computers, etc.) to result in operations implementing one or more of the described features. Similarly, computer systems are also described that can include one or more processors and one or more memories coupled to the one or more processors. A memory, which can include a computer-readable storage medium, can include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including but not limited to a connection over a network (e.g. the Internet, a wireless wide area network, a local area network, a wide area network, a wired network, or the like), via a direct connection between one or more of the multiple computing systems, etc.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system can include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user can be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital MRI image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and sub combinations of the disclosed features and/or combinations and sub combinations of several further features disclosed above. In addition, the logic flows depicted in the accompanying figures and/or described herein do not necessarily require the particular order shown, or sequential order, to achieve desirable results. Other implementations can be within the scope of the following claim.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: The Electrophysiology of a Human Obsession in Nucleus Accumbens

Microelectrode recordings during awake deep brain stimulation surgery for obsessive-compulsive disorder revealed robust brain oscillations, plainly visible throughout the ventral striatum. There was an elegant topological correspondence between each oscillation and the underlying brain anatomy, most prominently a ˜35 Hz gamma-oscillation specific to the nucleus accumbens. Direct provocation of the patient's contamination obsession modulated both firing rate and gamma-oscillation amplitude within the nucleus accumbens.

### Introduction

Have you ever gone back into your house shortly after leaving to make sure the oven was turned off, despite remembering turning it off? Have you then had the urge to check it yet again? These transient motivations are a normal part of the human experience that reinforce patterned behavior, and most of us can suppress them when they contradict what we know to be reasonable. But this ability to suppress is dysfunctional in those with obsessive-compulsive disorder (OCD), a neuropsychiatric disease characterized by repetitive physical or mental acts (compulsions) directed toward unwanted persistent images, thoughts, or impulses (obsessions) (1). The execution of compulsions consumes the time and effort of individuals to the degree that they dramatically interrupt personal and professional activities (2). Standard treatment is a combination of systematic exposure to the objects of obsession during cognitive behavioral therapy and medical intervention (3). Beginning 15 years ago, deep brain stimulation (DBS) emerged as a therapy for patients who fail the most aggressive standard treatment (4). As a region of confluent cortical, striatal and thalamic projections, the region of the nucleus accumbens (NAc) was felt to be an ideal initial target for DBS. Long-term studies of therapeutic outcome have substantiated its' efficacy in many patients (5-7). However, NAc DBS does not help some patients (8), and this can likely be attributed to variability in electrode positioning and individuals' functional anatomy. Variable response DBS (deep brain stimulation) for other diseases is partially mitigated by performing electrode implantation awake, making microelectrode recordings to identify neuronal populations whose activity correlates with disease-related tasks (e.g. limb movement in Parkinson's disease) (9). This strategy had remained unrealized for OCD until a patient of ours with a particular contamination obsession underwent awake NAc-DBS surgery. The provocable nature of his disease allowed for electrophysiological characterization of the fundamental processes that underlie obsessive thoughts.

### Methods and Materials

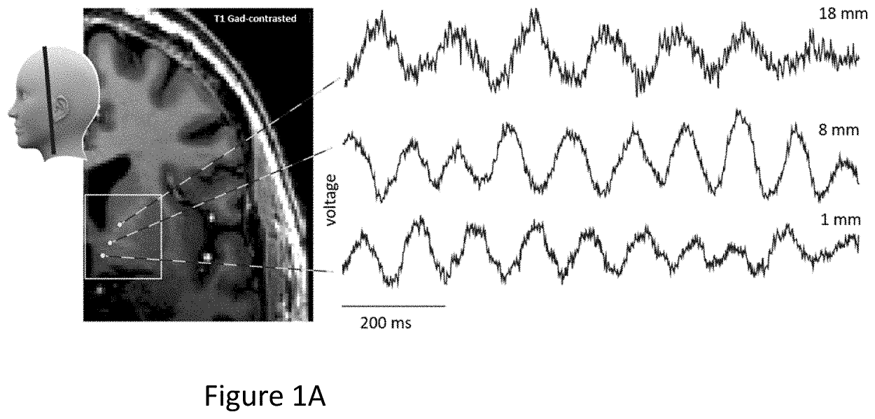

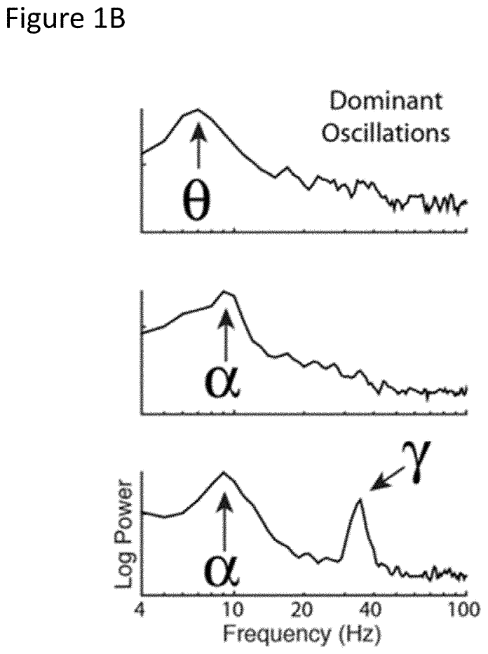

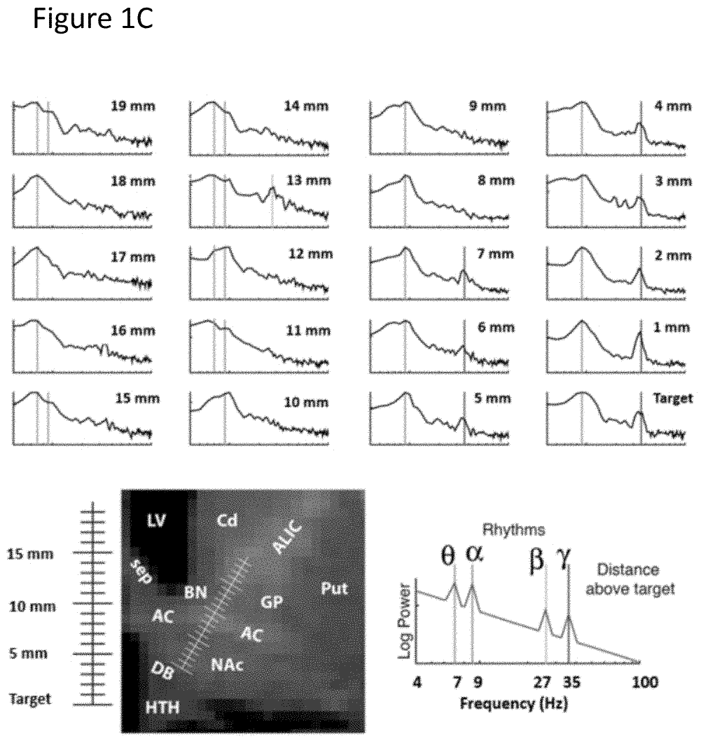

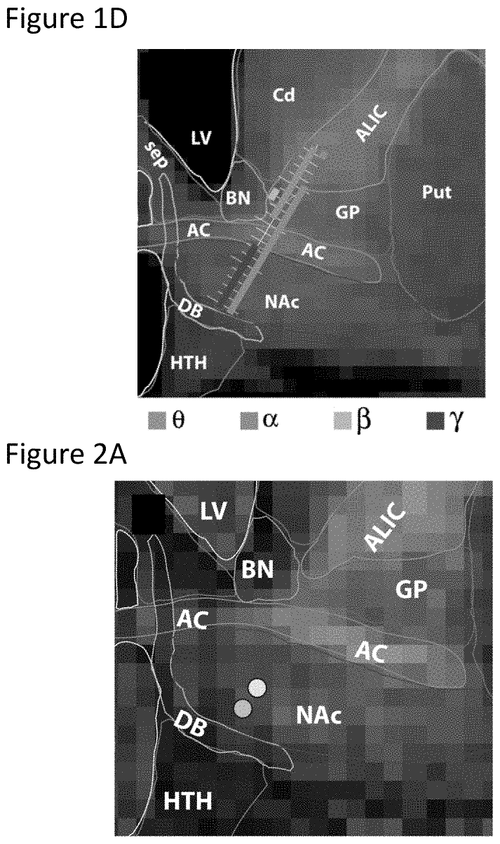

Patient and surgical implantation: A 64-year-old male patient with intractable obsessive-compulsive disorder, refractory to all medications, presented for bilateral deep brain stimulation electrode (DBS) implantation of the ventral capsule/ventral striatum, a region that includes the nucleus accumbens. His disease centered on cleanliness and bathroom-related activities, particularly brushing his teeth, causing marked impairment in his ability to carry out his normal activities of daily life. The patient consented to participate in a research protocol during the awake surgery for implantation of these leads. Stanford's internal review board approved the study and the consent process (IRB #33146). Stereotactic targeting and alignment to the left nucleus accumbens was performed with the NexFrame and Stealth S7 system (Medtronic, Minneapolis, Minn.). A cannula was stereotactically passed from the middle frontal gyrus to the ventro-medial internal capsule aligned to nucleus accumbens in-plane with the anterior commissure (FIG. 1-1). From the tip of the cannula, a microelectrode (0.5-1 MΩ platinum-iridium; FHC, Bowdoin, Me.) was advanced 20 mm to a target in the ventral nucleus accumbens (FIG. 1-1). The target location for the stereotactic placement in the AC-PC coordinate system was at x=6.0 mm, y=15.1 mm, z=−6.6 mm, with a trajectory of 34.2° from the midsagittal plane and 60.3° from the axial plane. With prolonged clinical stimulation, the patient achieved a 30% reduction in Yale-Brown obsessive-compulsive scale.

Signal analysis: Raw voltage, V° (t), was measured from the microelectrode, referenced to the cannula, and sampled at 50 kHz using a Guideline 3000 microelectrode recording system (Axon Instruments) (gain, 10,000; band-pass filtered from 1 Hz to 10 kHz), passed through a CyberAmp 380 amplifier/filter (Axon Instruments, Foster City, Calif.) (band-pass filtered from 1 Hz to 6 kHz), and sampled at 50,000 samples per s using a data acquisition interface (Power1401) and Spike software (version 2.7, Cambridge Electronic Design, Cambridge, England). Although previous studies were able to extract meaningful measurements of phase below 4 Hz (10), there was significant signal amplitude attenuation in this range, so we have limited our exploration in this study to frequencies above 4 Hz.

A number of steps were employed to isolate spikes from the raw voltage trace, as shown in 106 FIG. 1-1:

First, the raw voltage trace was high-pass filtered at 300 Hz,

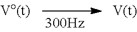

A linear threshold was then visually fit to the filtered voltage trace at each location to capture characteristic action potential voltage deflections (FIG. 1-1D).

Seven millisecond windows of data were obtained surrounding the sample of furthest excursion from baseline for each action potential deflection, τ_{q}. (FIG. 1-1G), from 2 ms prior to 5 ms after (e.g. \hat{V}_{q}(t′)=\hat{V}(t−τ_{q}), where −2 ms≤t′≤5 ms). The average of these windows gives the characteristic action potential shape (FIG. 1-1E).

These data windows surrounding action potential times were then decomposed with a principal component approach. A singular value decomposition is used to determine the eigenvalues λₖ and eigenvectors e̅ₖ̅ of the correlation matrix: C(t′, t″)=Σ_{q}\hat{V}_{q}(t′)\hat{V}_{q}(t″). Note that the baseline is, in effect, subtracted off of each window as a byproduct of the high-pass filtering. These eigenvectors, Ce̅ₖ̅ =λₖe̅ₖ̅ , reveal characteristic shapes in the temporal shape of the action potential that vary orthogonally, and are ordered by magnitude of corresponding eigenvalue: λ₁>λ₂> . . . >λ_{T} (where T≡number of time points in −2 ms to 5 ms interval). If we define the rotation matrix (k, t)=(e̅₁̅, e̅₂̅, . . . , e̅_̅{̅T̅}̅), then the projection, W(k, q), of each individual spike in the ensemble into the new eigenvector space is: W(k,q)=Σ_{t′}A(k,t′)\hat{V}_{q}(t′). The inverse rotation matrix A⁻¹ (where A⁻¹A=1) allows us to remove the weighted spike components (the first 3 eigenvectors) surrounding spike at time τ. from the raw voltage time series, leaving the local field potential (LFP): V(t′τ_{q})=V⁰(t′+τ_{q})−Σ_{k=1,2,3}A⁻¹(t′, k)W(k, q).

From this LFP, oscillations were characterized as follows:

Power spectral densities (PSDs) were calculated using Welch's averaged periodogram method, with 1 s windows, using a Hann window, stepping through (t) in 250 ms intervals (FIG. 1). Peaks in the PSDs were visually apparent above a 1/f background shape, centered at 7 Hz (theta), 9 Hz (alpha), 25 Hz (beta), and 36 Hz (gamma).

Rhythm amplitudes were calculated by band-passing the local field potential, V(t), using a 3rd order Butterworth filter for a specified frequency range, F, to obtain the “bandlimited” potential, V(F,t). A complex analytic signal, V̅(F,t)=V(F,t)+iV^{lM}(F,t) was constructed using the Hilbert transform, which can also be expressed in polar notation as V̅(F,t)=r(F,t)e^{iϕ(F,t)}. The rhythm amplitudes plotted with bars in FIG. 3 are the averages of (F,t) in 1 s blocks. In this study, the alpha range is F→8-10 Hz, and the gamma range is F→31-39 Hz.

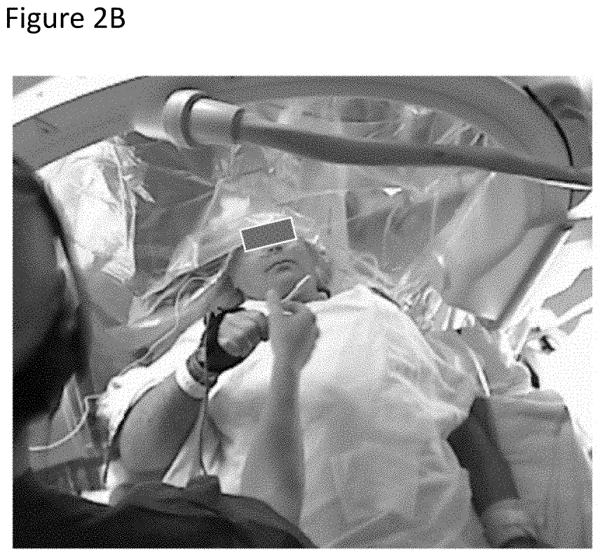

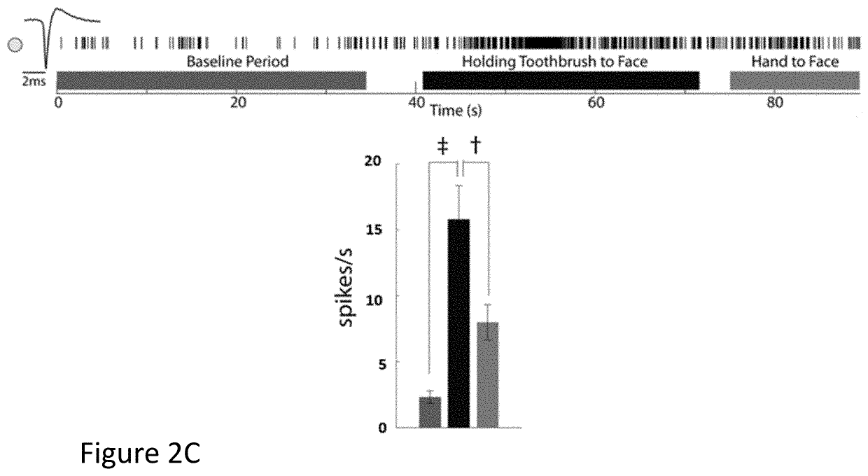

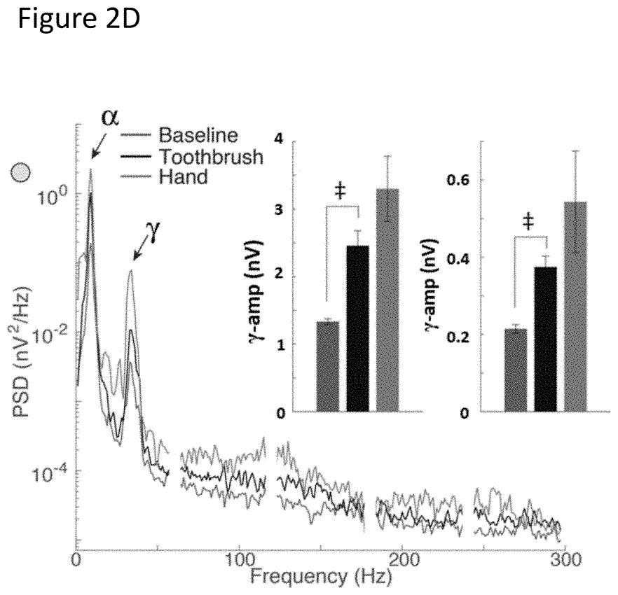

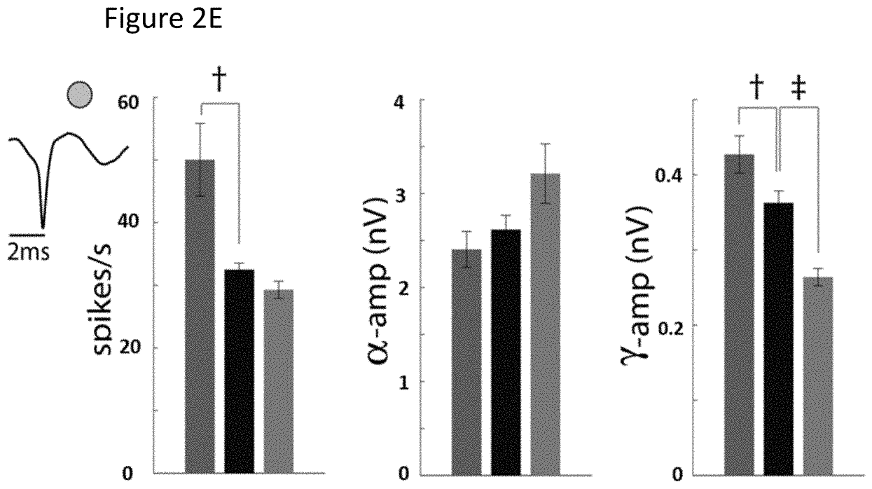

Anatomic localization: As illustrated in FIG. 1-2, microelectrode recording position was determined by fusion of the post-surgical CT to the pre-surgical MRI, using a normalized mutual information approach, and reslicing in-plane with the DBS shank while preserving midline symmetry. Then the intraoperative microelectrode recording positions were inferred from the corresponding post-implantation DBS electrode lead positions (where the terminal lead was at the target position). Grey-matter nulled MR, white-matter nulled MR, and T1-post gadolinium contrasted were overlaid, so underlying ventral striatal anatomic structures could be clearly delineated.

Compulsion provocation: A simple provocation test was designed based on a self-reported fear known to trigger his compulsive full body cleaning. After a brief baseline period, a psychiatrist at the bedside (N. W.) handed the patient a toothbrush, telling him first to bring it to his face, and then told to “imagine brushing your teeth with this dirty toothbrush”. The toothbrush was then taken back from the patient, and, as a control, the patient was then instructed to bring his hand back to his face without the toothbrush (FIG. 2). This provocation testing was performed at 3 mm and 1 mm above target, where actively spiking neurons were identified.

### Results

A map of human brain oscillations in the ventral capsule/ventral striatal region: Field potentials measured were measured at every millimeter from the opening of a stereotactic guidance cannula to the NAc target 2 cm below (FIG. 1). The raw potential traces showed visually apparent oscillations, plainly reflected by peaks in the power spectral densities (PSD). When these PSDs are viewed alongside one another, a clear topological relationship between oscillatory frequency and brain anatomy emerges:

A robust 35 Hz-centered gamma oscillation (hγ₃₅) was found specifically in the NAc and nowhere else. Based upon comparison with recent human segmentations using diffusion tractography (12), our NAc recordings are most likely in the shell sub region.

7 Hz-centered theta oscillations extended throughout the recorded portion of the anterior limb of the internal capsule (ALIC), including where capsular fibers were co-localized with the globus pallidus (GP) and the bed nucleus of the stria terminalis (BNST).

9 Hz-centered alpha oscillations were present throughout all structures except for the ALIC.

A small focus of 27 Hz-centered beta oscillation was found at the confluence of ALIC, GP, and BNST, making it difficult to attribute to a single structure.

Physiological changes during provocation of an obsessive fear: As the microelectrode tip neared the ventral NAc target, the patient was handed a toothbrush and told to bring it to his face, and then to imagine it dirty while also imagining brushing his teeth with it. This test was performed for clinical purposes—to attempt neuronal action potential modulation correlated with his contamination obsession and confirm regional involvement of his disease, much like sensorimotor testing is used in movement disorders (13). Robustly firing neurons were studied at two sites within NAc, 2 mm from one another, and 4&6 mm ventral to the dorsal border (FIG. 2). In response to the provocation test, we made the following observations:

At the more dorsal NAc site, firing rate of the measured neuron increased specifically during provocation of the compulsion. The amplitude of both the alpha and gamma oscillations increased with provocation (FIG. 2, Supplemental video).

Conversely, at the more ventral NAc site, firing rate of an isolated neuron as well as gamma amplitude decreased with provocation of the compulsion. There was significant correlation between gamma oscillation amplitude and spike rate during the pre-provocation period, and not for the periods during or following provocation with the toothbrush (FIG. 2-1).

### Discussion

This NAc-specific hγ₃₅ oscillation implies a common physiological element amongst NAc microcircuits, which are known to be composed of medium spiny neurons (MSNs) and a variety of different classes of interneurons. The observation that obsession provocation induces opposite hγ₃₅-amplitude responses at different NAc sites implies that this common element is present across different NAc microcircuit types. In rats, a ˜50 Hz gamma oscillation (rγ₅₀) is present in NAc, and not the remainder of the striatum (14). Using pharmacological manipulation, it was shown that rγ₅₀ is specifically attributable to subthreshold fluctuations in the membrane potential of parvalbumin-positive GABAergic fast-spiking interneurons (FSIs) (15). The rat rγ₅₀ may help us interpret the human hγ₃₅ if, as we hypothesize, both emerge from genetically homologous FSIs that diverged during evolution, resulting in slightly different timescales of intrinsic subthreshold membrane fluctuation. Careful measurement showed that some NAc rγ₅₀ are coherent with select sites in prefrontal cortex, piriform cortex, and the hippocampus (16). Assuming hγ₃₅-rγ₅₀ homology, hγ₃₅ coherence might reveal NAc interactions with these other brain areas in the human, which could be used as a tool for paired stimulation in neurosurgical intervention. One might speculate that these oscillations actually facilitate information transfer between brain regions, beyond serving only as a signature of interaction, but that cannot be established from this case alone. Measurements from DBS macroelectrodes in the NAc found a similar ˜10 Hz oscillation during task engagement, but nothing consistent with the hγ₃₅ in the signal (17, 18). This discrepancy might be explained if the microcircuit motif that generates a 35 Hz isn't coherent across a large enough volume to be picked up by the DBS macroelectrode, which fits with the observation that the hγ₃₅ motif has conjugate changes in sites separated by just 2 mm (FIG. 2).

The finding that provocation of the patient's contamination obsession induced physiological changes in NAc is an initial step forward to better understand how obsessions are processed in the human brain. In isolation, differential action potential firing activity at different sites within NAc could be attributed to the capture of different neuron types within a functionally isotropic region. However, local field potentials reflect a property of the local ensemble of neurons and were also opposite in the magnitude of their shift with provocation, suggesting that the different electrophysiological responses we observed within the NAc reflect distinct microcircuits with different functions. Likely, our two conjugate responses to compulsive fear are from different types of microcircuits that share the hγ₃₅-FSI type, and may be related to different MSN types (e.g. D1 vs D2 dopamine receptors) (19). There are distinct MSN-based microcircuits in the rat NAc shell, which are topologically organized differentially by positive- and negative-motivational-valence (20). In light of this, differential NAc responses we observed may reveal conjugate motivational valence microcircuits, with obsession triggered increased firing rate and hγ₃₅-amplitude more dorsally, and the physiological converse 2 mm beneath. During intraoperative stimulation testing, our patient began smiling with an outwardly euphoric affect, while verbally stating this distressed him, which may have been induced by simultaneous stimulation of multiple accumbens microcircuit types (21). Although NAc involvement in a brain circuit underlying OCD has been demonstrated with functional imaging (22) and inferred by clinical improvement with NAc-DBS (4, 6, 21), this case shows directly that provocation of an obsession is associated with changes in firing rate and LFP oscillatory power in human NAc.

### REFERENCES

1. Westphal CFO (1877): Ueber Zwangsvorstellungen. Berliner Klinische Wochenschrift. 46:669-672.

2. American Psychiatric Association (2013): Diagnostic and statistical manual of mental disorders (DSM-5®). American Psychiatric Pub.

3. Grant J E (2014): Obsessive-compulsive disorder. New England Journal of Medicine. 371:646-653.

4. Sturm V, Lenartz D, Koulousakis A, Treuer H, Herholz K, Klein J, et al. (2003): The nucleus accumbens: a target for deep brain stimulation in obsessive-compulsive-and anxiety-disorders. Journal of chemical neuroanatomy. 26:293.

5. Alonso P, Cuadras D, Gabriels L, Denys D, Goodman W, Greenberg B D, et al. (2015): Deep brain stimulation for obsessive-compulsive disorder: a meta-analysis of treatment outcome and predictors of response. PloS one. 10:e0133591.

6. Fayad S M, Guzick A G, Reid A M, Mason D M, Bertone A, Foote K D, et al. (2016): Six-nine year follow-up of deep brain stimulation for obsessive-compulsive disorder. PloS one. 11:e0167875.

7. Sheth S A, Neal J, Tangherlini F, Mian M K, Gentil A, Cosgrove G R, et al. (2013): Limbic system surgery for treatment-refractory obsessive-compulsive disorder: a prospective long-term follow-up of 64 patients. Journal of neurosurgery. 118:491-497.

8. Mian M K, Campos M, Sheth S A, Eskandar E N (2010): Deep brain stimulation for obsessive-compulsive disorder: past, present, and future. Neurosurgical focus. 29:E10.

9. Romanelli P, Heit G, Hill B C, Kraus A, Hastie T, Bronte-Stewart H M (2004): Microelectrode recording revealing a somatotopic body map in the subthalamic nucleus in humans with Parkinson disease. Journal of neurosurgery. 100:611-618.

10. Wu H, Miller K J, Blumenfeld Z, Williams N R, Ravikumar V K, Lee K E, et al. (2018): Closing the loop on impulsivity via nucleus accumbens delta-band activity in mice and man. Proceedings of the National Academy of Sciences. 115:192-197.

11. Miller K J, Sorensen L B, Ojemann J G, den Nijs M (2009): Power-Law Scaling in the Brain Surface Electric Potential. PLOS Computational Biology. 5:e1000609.

12. Baliki M N, Mansour A, Baria A T, Huang L, Berger S E, Fields H L, et al. (2013): Parceling human accumbens into putative core and shell dissociates encoding of values for reward and pain. Journal of Neuroscience. 33:16383-16393.

13. Benabid A-L, Krack P, Benazzouz A, Limousin P, Koudsie A, Pollak P (2000): Deep brain stimulation of the subthalamic nucleus for Parkinson's disease: methodologic aspects and clinical criteria. Neurology. 55:S40-44.

14. Berke J D, Okatan M, Skurski J, Eichenbaum H B (2004): Oscillatory entrainment of striatal neurons in freely moving rats. Neuron. 43:883-896.

15. Bracci E, Centonze D, Bernardi G, Calabresi P (2003): Voltage-dependent membrane potential oscillations of rat striatal fast-spiking interneurons. The Journal of physiology. 549:121-130.

16. Berke J (2009): Fast oscillations in cortical-striatal networks switch frequency following rewarding events and stimulant drugs. European Journal of Neuroscience. 30:848-859.

17. Cohen M X, Axmacher N, Lenartz D, Elger C E, Sturm V, Schlaepfer T E (2009): Good vibrations: cross-frequency coupling in the human nucleus accumbens during reward processing. Journal of cognitive neuroscience. 21:875-889.

18. Cohen M X, Axmacher N, Lenartz D, Elger C E, Sturm V, Schlaepfer T E (2009): Nuclei accumbens phase synchrony predicts decision-making reversals following negative feedback. Journal of Neuroscience. 29:7591-7598.

19. Graziane N M, Sun S, Wright W J, Jang D, Liu Z, Huang Y H, et al. (2016): Opposing mechanisms mediate morphine-and cocaine-induced generation of silent synapses. Nature neuroscience. 19:915.

20. Reynolds S M, Berridge K C (2002): Positive and negative motivation in nucleus accumbens shell: bivalent rostrocaudal gradients for GABA-elicited eating, taste “liking”/“disliking” reactions, place preference/avoidance, and fear. Journal of Neuroscience. 22:7308-7320.

21. Haq I U, Foote K D, Goodman W G, Wu S S, Sudhyadhom A, Ricciuti N, et al. (2011): Smile and laughter induction and intraoperative predictors of response to deep brain stimulation for obsessive-compulsive disorder. Neuroimage. 54:S247-S255.

22. Figee M, Vink M, de Geus F, Vulink N, Veltman D J, Westenberg H, et al. (2011): Dysfunctional-reward circuitry in obsessive-compulsive disorder. Biological psychiatry. 69:867-874.

What is claimed is:

1. A method of identifying the anatomic structure of a nucleus accumbens in a subject, said method comprising:
(i) inserting an electrode into the ventral striatum of a subject; and
(ii) detecting an oscillatory frequency of 30-40 Hz, thereby identifying the anatomic structure of a nucleus accumbens in said subject.

2. The method of claim 1 wherein said oscillatory frequency is a frequency of about 35 Hz.

3. The method of claim 1, wherein said electrode is a microelectrode.

4. The method of claim 1, further comprising detecting a theta oscillatory frequency or an alpha oscillatory frequency.

5. The method of claim 1, wherein said subject is suffering from obsessive compulsive disorder.

6. The method of claim 1, comprising after said detecting administering an electrical stimulation to the nucleus accumbens of said subject.

7. The method of claim 6, wherein a dose of said electrical stimulation is less than a dose corresponding to deep brain stimulation.

8. The method of claim 6, wherein said electrical stimulation is administered at a frequency of 5 Hz, 10 Hz, 12 Hz, 160 Hz, 212 Hz, or 333 Hz.

9. The method of claim 6, wherein said electrical stimulation is administered at 100 milliseconds, 1 minute, 15 minutes, or 1 hour.

10. A method of treating an obsessive compulsive disorder (OCD) subject, said method comprising:
detecting an oscillatory frequency of 30-40 Hz in the ventral striatum of an OCD subject; and
(ii) administering an electrical stimulation to the nucleus accumbens of said OCD subject in response to said oscillatory frequency, thereby treating said OCD subject.

11. The method of claim 10, wherein said oscillatory frequency is a frequency of about 35 Hz.

12. The method of claim 10, further comprising detecting a theta oscillatory frequency or an alpha oscillatory frequency.

13. The method of claim 10, wherein a dose of said electrical stimulation is less than a dose corresponding to deep brain stimulation.

14. The method of claim 10, wherein said electrical stimulation is administered at a frequency of 5 Hz, 10 Hz, 12 Hz, 160 Hz, 212 Hz, or 333 Hz.

15. The method of claim 10, wherein said electrical stimulation is administered at 100 milliseconds, 1 minute, 15 minutes, or 1 hour.
